(19) United States
(12) Patent Application Publication
(10) Pub. No.: US 2013/0345504 A1
(43) Pub. Date: Dec. 26, 2013

(54) REUSABLE IN-VIVO DEVICE, SYSTEM AND METHOD OF ASSEMBLY THEREOF

(71) Applicants: Nachum Shamir, Atlanta, GA (US); Zvika Gilad, Hod Hacarmel (IL)

(72) Inventors: Nachum Shamir, Atlanta, GA (US); Zvika Gilad, Hod Hacarmel (IL)

(21) Appl. No.: 14/003,849

(22) PCT Filed: Mar. 16, 2012
(86) PCT No.: PCT/US12/29382

§ 371 Date: Sep. 9, 2013

Related U.S. Application Data
(60) Provisional application No. 61/453,816, filed on Mar. 17, 2011.

(51) Int. Cl. A61B 1/04; A61B 1/00

(52) U.S. Cl. CPC A61B 1/041; A61B 1/0011
USPC 600/104; 600/109; 29/428

(57) ABSTRACT

Reusable devices, systems and methods of assembly of reusable devices comprise an inner portion designed to be reused and an outer portion designed to be disposed. The reusable inner portion is a closed, self-contained receptacle comprising a housing, which encapsulates at least an imaging device, at least one illumination source, at least one rechargeable battery, and a controller. A first end of the closed reusable inner portion is covered by an optical system, and a second end of the closed reusable inner portion comprises battery contacts. The disposable outer portion comprises at least a shell and an optical dome. The housing of the reusable inner portion is inserted into the shell, and the optical dome is attached to the shell so as to detachably seal the reusable inner portion of the in-vivo device within the disposable outer portion of the device.

### FIELD OF THE INVENTION

This application relates to the field of reusable in-vivo devices and systems, and methods of assembling reusable in-vivo devices.

### BACKGROUND OF THE INVENTION

Swallowable capsule endoscopes are well-known devices that are used in order to acquire images of in-vivo lumens, e.g., the gastrointestinal (GI) tract. Nowadays, there is an on-going need for adding maneuvering capabilities to such capsules and to other in-vivo devices, and to thus provide the ability to perform various in-vivo operations at specific locations along the lumen that such devices travel. One method of maneuvering an in-vivo device while in-vivo is to use external magnetic fields, which may surround the patient being examined and/or treated by the in-vivo device and may control the location of the in-vivo device.

An in-vivo device that may be externally controlled with regard to its in-vivo location and with regard to operations that the device may perform while in-vivo might be quite expensive. Furthermore, in-vivo devices, even those without maneuvering capabilities, e.g., autonomous capsule endoscopes, might also be expensive to the end-user.

There is a need for an in-vivo device that may not be expensive to the end-user and that may be easy to assemble, in order to encourage use of such a device for the detection and/or treatment of various malignancies and pathologies that may be present in-vivo.

### SUMMARY OF THE INVENTION

The present invention provides reusable in-vivo devices and systems and methods for assembling such reusable in-vivo devices, which may assist in lowering the price of these devices to the end-user. These devices should be substantially easily retrievable from within the patient, such that they could be reused for an additional operation, thereby maintaining a relatively low price.

In some embodiments, the reusable in-vivo device may comprise a portion that may be considered reusable or able to be reused (the “reusable” portion) and a portion that may be considered disposable and not intended to be reused (the “disposable” portion). In some embodiments, the reusable portion may comprise a housing. The housing may encapsulate an imaging device, an optical system, an illumination source and a controller. In some embodiments, the disposable portion may comprise at least a shell and an optical dome. According to some embodiments, the housing of the reusable portion may be inserted into the shell, and the optical dome may be attached to the shell so as to detachably seal the reusable portion of the in-vivo device within the disposable portion of the device.

In some embodiments, the reusable portion may be a closed, self-contained receptacle comprising a housing. The housing may encapsulate an imaging device, at least one illumination source, at least one rechargeable battery, and a controller. A first end of the closed reusable portion may be covered by an optical system, and a second end of the closed reusable portion may comprise battery contacts.

According to some embodiments, the reusable portion may further comprise operational tools for performing various operations in-vivo.

In some embodiments, the reusable portion may comprise at least one rechargeable battery. In other embodiments, the disposable portion may comprise at least one disposable battery.

In some embodiments, the optical dome may be detachably attached to the shell by means selected from a group consisting of: friction fit, screw threading, or a combination thereof. In some embodiments, the means for detachably attaching the optical dome to the shell may comprise an O-ring for sealing the optical dome to the shell. In some embodiments, the reusable portion may further encapsulate a magnetic element for enabling maneuvering of the reusable device using magnetic fields.

In some embodiments, a reusable in-vivo device may comprise a reusable device and a recharging unit. The reusable device may comprise a closed, self-contained reusable portion and a disposable portion. The reusable portion may comprise a housing, which may encapsulate an imaging device, an optical system, an illumination source, a rechargeable battery, and a controller. The disposable portion may comprise at least a shell and an optical dome. In some embodiments, the housing of the reusable portion may be inserted into the shell, and the optical dome may be attached to the shell so as to detachably seal the reusable portion of the in-vivo device within the disposable portion of the device. In some embodiments, the recharging unit may be for recharging the rechargeable battery encapsulated within the reusable portion, thereby enabling reuse of the reusable device.

In some embodiments, a method of assembling a reusable in-vivo device may comprise the step of retrieving a reusable in-vivo device from within a patient. The reusable device may comprise an inner portion that is intended to be reused (the reusable portion) and an outer portion that is intended to be disposed (the disposable portion). According to some embodiments, the reusable inner portion may encapsulate an imaging device, an optical system, an illumination source and a controller, and the disposable outer portion may comprise at least a shell and an optical dome. The method may further comprise the steps of removing the reusable inner portion of the device from within the shell and optical dome, and placing said reusable inner portion into a second disposable outer portion. In some embodiments, the second disposable outer portion may comprise at least a second shell and a second optical dome. The method may comprise the step of detachably sealing the reusable inner portion within the second disposable outer portion.

In some embodiments, the inner reusable portion may be a closed, self-contained receptacle comprising a housing. The housing may encapsulate an imaging device, at least one illumination source, at least one rechargeable battery, and a controller. A first end of the closed reusable portion may be covered by an optical system, and a second end of the closed reusable portion may comprise battery contacts.

In some embodiments, the method may comprise the step of detaching the shell of the device from the optical dome of the device, prior to removing the inner portion from within the shell and dome.

In some embodiments, the method may comprise the step of recharging at least one battery that may be encapsulated within the reusable inner portion, prior to placing the reusable inner portion into the second disposable outer portion. In other embodiments, the method may comprise the step of inserting at least one disposable battery into the second disposable outer portion prior to placing the reusable inner portion into the second disposable outer portion.

In some embodiments, the method may comprise the step of inserting the detachably sealed reusable in-vivo device into a patient following the step of detachably sealing the reusable inner portion within the second disposable outer portion. In some embodiments, the method may comprise the step of maneuvering the reusable in-vivo device through a lumen of a patient, and then retrieving the in-vivo device from within the patient. In some embodiments, the method may further comprise the step of inserting the detachably sealed device into a patient, following by the step of maneuvering the reusable in-vivo device through the patient's lumen.

### BRIEF DESCRIPTION OF THE DRAWINGS

The above and other objects and advantages of the invention will be apparent upon consideration of the following detailed description, taken in conjunction with the accompanying drawings, in which the reference characters refer to like parts throughout and in which:

FIG. 1 illustrates a schematic front-side perspective view of a reusable portion of an in-vivo device, in accordance with one embodiment of the present invention;

FIG. 2 illustrates a schematic back-side perspective view of a reusable portion of an in-vivo device, in accordance with one embodiment of the present invention;

FIGS. 3A-3C illustrate a schematic side-view, a schematic transparent view and a schematic cross-sectional view, respectively, of a reusable portion of an in-vivo device, in accordance with one embodiment of the present invention;

FIG. 4 illustrates a schematic back-side exploded perspective view of a reusable in-vivo device, in accordance with one embodiment of the present invention;

FIG. 5 illustrates a schematic back-side perspective view of a detachably assembled reusable in-vivo device, in accordance with one embodiment of the present invention;

FIGS. 6A-6B illustrate a schematic view of a recharging unit before and during recharging of a reusable portion of an in-vivo device, respectively, in accordance with one embodiment of the present invention;

FIG. 7 illustrates a schematic front-side perspective view of a reusable portion of an in-vivo device, in accordance with another embodiment of the present invention;

FIG. 8 illustrates a schematic front-side exploded perspective view of a reusable in-vivo device, in accordance with an embodiment of the present invention;

FIG. 9 illustrates a schematic front-side perspective view of a detachably assembled reusable in-vivo device, in accordance with an embodiment of the present invention;

FIG. 10 illustrates a schematic side view of a detachably assembled reusable in-vivo device, in accordance with an embodiment of the present invention;

FIG. 11 illustrates a schematic view of a recharging unit during recharging of a reusable portion of an in-vivo device, in accordance with an embodiment of the present invention; and

FIG. 12 illustrates a method of assembling a reusable in-vivo device, in accordance with an embodiment of the present invention.

It will be appreciated that for simplicity and clarity of illustration, elements shown in the figures have not necessarily been drawn to scale. For example, the dimensions of some elements may be exaggerated relative to other elements for clarity. Further, where considered appropriate, reference numerals may be repeated among the figures to indicate corresponding or analogous elements.

### DETAILED DESCRIPTION OF THE INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

The reusable in-vivo devices, systems, and methods for assembling such reusable devices as described below provide examples of devices that are easy to assemble and reassemble following a substantially easy retrieval from within a patient's body. These devices may include various operational tools for performing all sorts of in-vivo operations once these devices reach a location of interest.

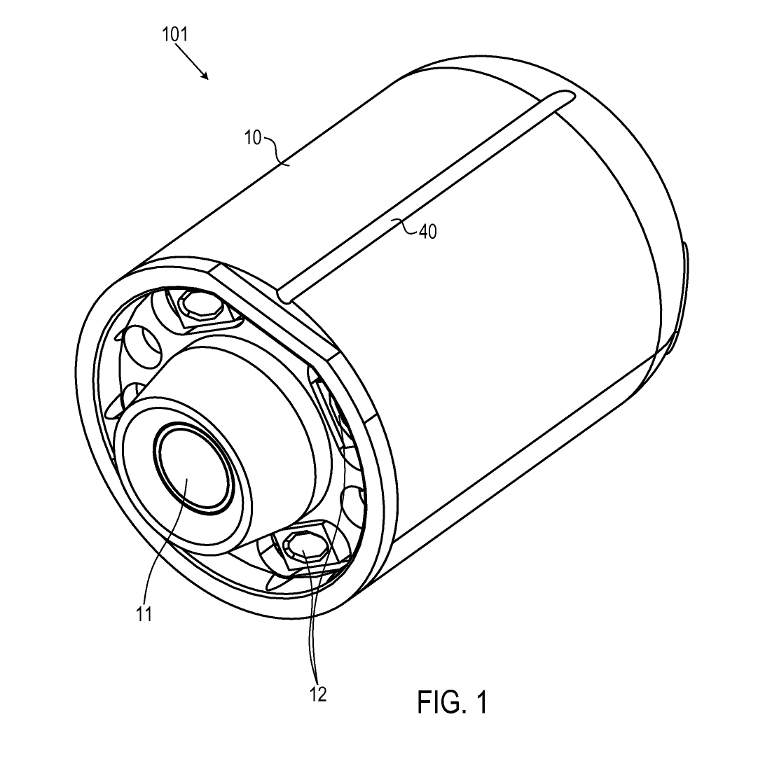

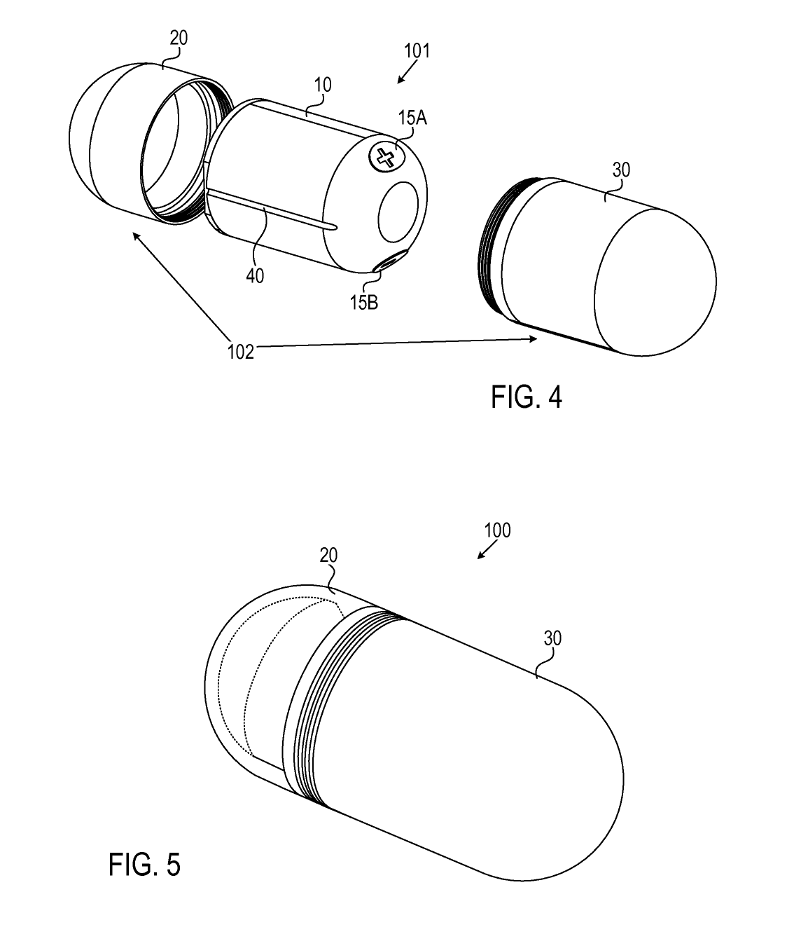

Reference is now made to FIG. 1, which illustrates a schematic front-side perspective view of an inner portion of an in-vivo device, which may be considered or is intended to be reusable, in accordance with one embodiment of the present invention. The reusable inner portion 101 may comprise a housing 10, which may have a shape of a cylinder, a sphere, a rectangle or any other shape that may later be easily inserted into an outer portion 102, which may be or is intended to be disposed after use (i.e., is disposable) (such as shown in FIG. 4). According to some embodiments, the outer portion 102 may have a shape that should be easy to place into and travel through a patient's lumen, e.g., by swallowing or by being inserted in-vivo using a delivery device. In some embodiments of reusable portion 101, housing 10 may encapsulate at least one illumination source 12, as well as optical system 11. Optical system 11 may comprise a plurality of lenses. The lenses may be encapsulated within housing 10 while being held in a lens holder. According to some embodiments, the lens holder may protrude outward from one of the ends of housing 10, thus some of the lenses may partially protrude outwards from housing 10 as well.

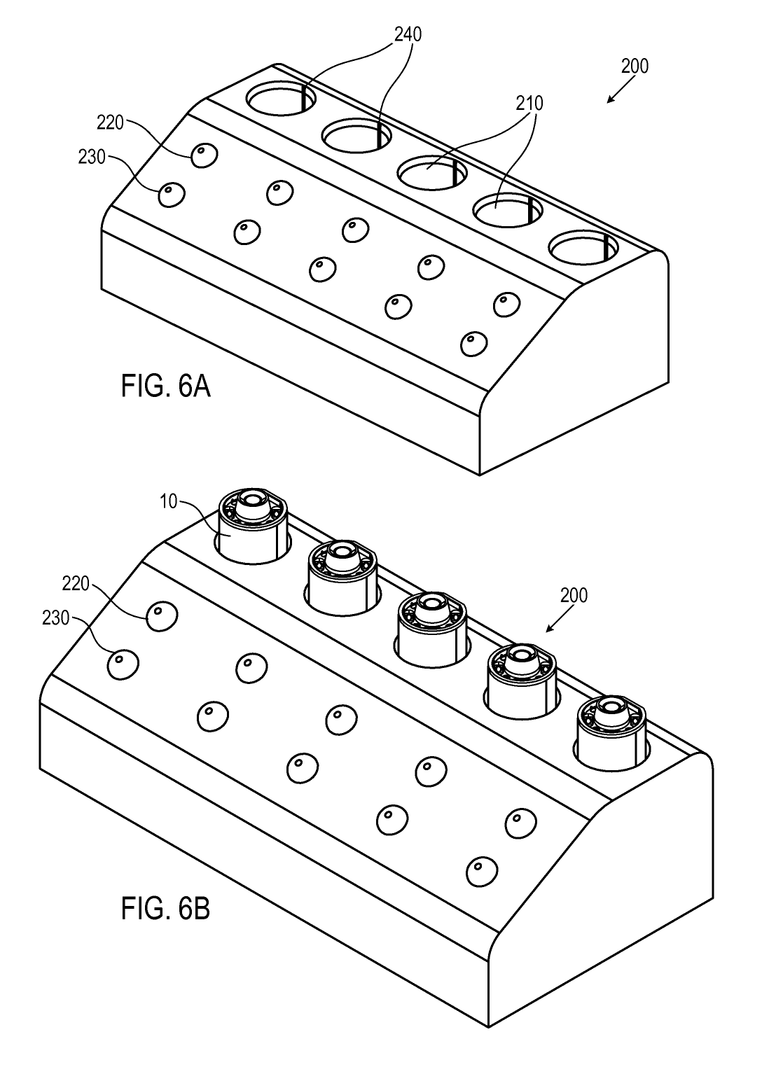

In some embodiments, reusable inner portion 101 may comprise a groove 40 formed into its outer surface. Groove 40 may extend along substantially the entire longitudinal axis of housing 10 of reusable portion 101, as shown in FIG. 1. In other embodiments, groove 40 may only partially extend along the longitudinal axis of housing 10. In yet other embodiments, groove 40 may extend along other positions of housing 10 of reusable portion 101, e.g., along part of the transverse axis of housing 10. As will be described later with regards to FIGS. 6A-6B, groove 40 may be used to place reusable portion 101 into recharging unit 200 at a specific orientation.

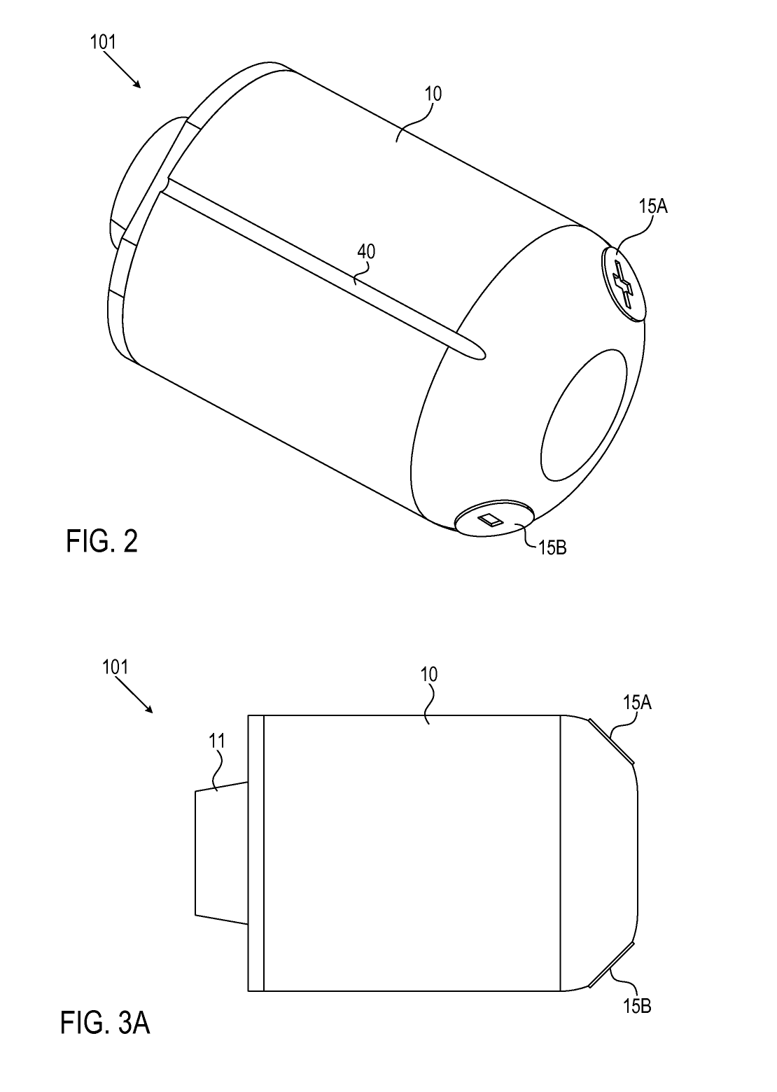

Reference is now made to FIG. 2, which illustrates a schematic back-side perspective view of a reusable portion of an in-vivo device, in accordance with one embodiment of the present invention. According to some embodiments, housing 10 of the reusable portion 101 may comprise or encapsulate at least one rechargeable battery. In order to recharge the rechargeable battery or a plurality of rechargeable batteries, while avoiding the need to disassemble the housing 10 of the reusable portion 101, housing 10 may comprise electrical contacts 15a and 15b. Electrical contacts 15a and 15b may be located opposite the typically protruding lens holder. Electrical contacts 15a and 15b may be used to connect between the rechargeable batteries that are encapsulated within housing 10 and a recharging device 200 (such as shown in FIGS. 6A-6B), in order to enable passage of energy from the recharging unit 200 to the batteries within housing 10.

However, in other embodiments, the reusable portion 101 or its housing 10 need not include rechargeable batteries. Instead, disposable batteries may be inserted into the device while assembling or reassembling the reusable portion 101 into the disposable portion 102. For example, disposable portion 102 may comprise at least one disposable battery such that when disassembling the reusable portion 101 from the disposable portion 102, the disposable portion 102 may be discarded along with the disposable battery(ies). A disposable portion 102 comprising a new set of disposable battery(ies) may then be assembled with the reusable portion 101, and battery contacts 15a and 15b may electrically connect between the disposable battery(ies) and the electrical components that may be encapsulated within the reusable portion 101.

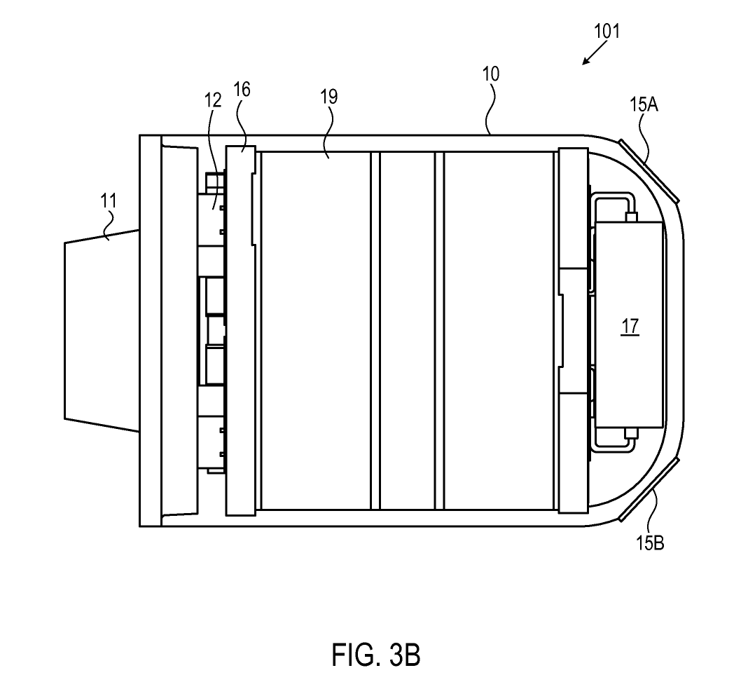

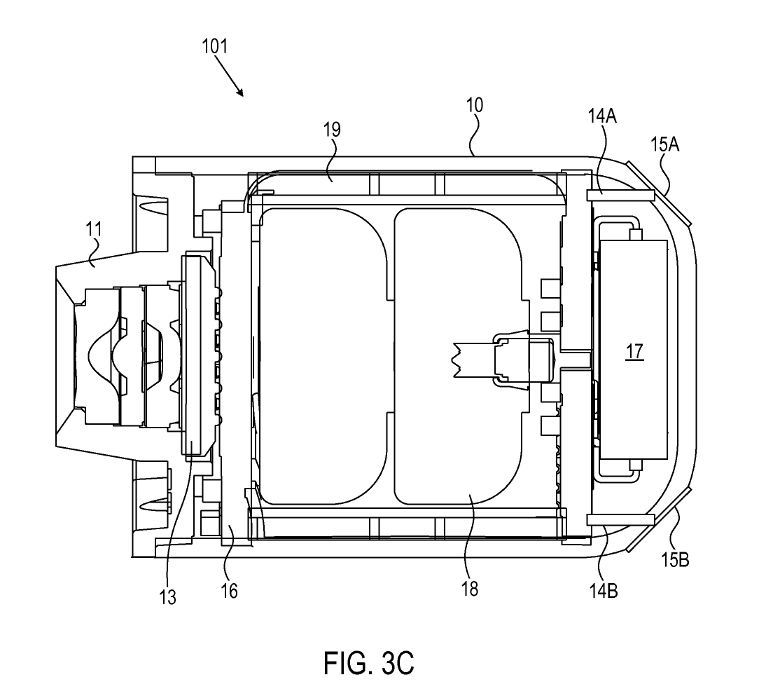

Reference is now made to FIGS. 3A-3C, which illustrate a schematic side-view, a schematic transparent view and a schematic cross-sectional view, respectively, of a reusable portion of an in-vivo device, in accordance with one embodiment of the present invention. FIG. 3A illustrates housing 10 of reusable portion 101. Housing 10 may comprise a lens holder portion of an optical system 11, which may protrude from one of the ends of housing 10. The other end of housing 10 may comprise at least two electrical contacts 15a and 15b. In some embodiments, the reusable device may comprise rechargeable batteries, such that the electrical contacts 15a and 15b may enable recharge of rechargeable batteries encapsulated within housing 10, e.g., electrical contacts 15a and 15b may electrically connect between the rechargeable batteries and the recharging unit 200. In other embodiments, the reusable device may comprise disposable batteries, such that electrical contacts 15a and 15b may provide an electrical contact between the electrical components within housing 10 and the disposable batteries positioned beside them, while both housing 10 and the disposable batteries may be placed within the disposable portion 102.

FIG. 3B illustrates a schematic transparent view of reusable portion 101 illustrated in FIG. 3A. FIG. 3B illustrates some of the components that may be encapsulated within housing 10. In some embodiments, housing 10 may comprise optical system 11, which may comprise a lens holder protruding outward from one of the ends of housing 10. Housing 10 may further comprise at least one illumination source 12 (although other numbers of illumination sources may be placed elsewhere within housing 10). According to some embodiments, both the at least one illumination source and the optical system 11 may be positioned or attached to a PCB 16. PCB 16 may be a rigid-flex or a full flex type PCB, though other PCB types may be used. Housing 10 may further encapsulate a switch 17, which may act as an on/off switch for activating or deactivating the reusable device. Switch 17 may be a Radio Frequency (RF) switch, which is a switch operated by an RF signal. Other types of on/off switches may be used.

According to some embodiments, housing 10 may comprise a magnetic element 19, which may be used to enable maneuvering of the reusable device using magnetic fields. One way to maneuver a device in-vivo may be by use of magnetic fields applied externally to a patient who may administer the device. In order for external magnetic fields to be able to control the location of the device, e.g., to move the device from one in-vivo location to another, the device typically encapsulates a magnetic element, e.g., magnetic element 19. When such a reusable device is controllable, it may include various operational tools for performing various examinations and/or treatments while it is in-vivo. For example, a device may comprise a medicament, and a controller may control release of the medicament at a specific location in-vivo, towards which the device may be maneuvered.

In other embodiments, the reusable device need not comprise a magnetic element, and may be maneuvered by different means, e.g., the device may comprise a motor. In yet other embodiments, the reusable device need not be maneuverable at all but rather may move passively along the body lumen.

FIG. 3C illustrates a cross-sectional view of the reusable portion 101 of the reusable in-vivo device. FIG. 3C illustrates components that housing 10 may comprise in addition to the components illustrated in FIG. 3B. In some embodiments, housing 10 may encapsulate an imaging device 13 that may be used to acquire in-vivo images, while the device passes along the body lumen, e.g., the gastrointestinal tract. As can be seen in FIG. 3C, the optical system 11 may comprise a lens holder, which may be used to hold a plurality of lenses. In some embodiments, illumination source(s) 12 may illuminate the lumen, and light reflected off the tissue of the lumen wall may be focused by the plurality of lenses held in optical system 11, onto the imaging device 13.

In some embodiments, housing 10 may comprise rechargeable batteries 18, which may be recharged in a recharging unit 200 (such as shown in FIGS. 6A-6B) through electrical contacts 15a and 15b. Electrical contacts 15a and 15b may be connected to PCB 16 through contact wires or contact springs 14a and 14b, respectively. In some embodiments, housing 10 may comprise a power induction unit (not shown) that may receive power from an external source. The power induction unit may comprise an electric coil for inducing therein an alternating power signal by an external electromagnetic field generator or by an external radio frequency (“RF”) transmitter. The power induction unit may also include a rectifier for rectifying the induced power signal, and for recharging rechargeable batteries 18. In other embodiments, the reusable device may comprise disposable batteries, which may be inserted into the device once it is reassembled. In some embodiments, the disposable batteries may be an inseparable part of the disposable portion 102, and thus there is no need for insertion of the disposable batteries into the device during a separate step.

According to some embodiments, reusable portion 101 may be a closed self-contained receptacle, which encapsulates several components, such as PCB 16, switch 17, magnetic element 19, imaging device 13, illumination sources 12, and rechargeable batteries 18. Other or additional components may be carried within reusable portion 101. A first end of reusable portion 101 may be covered by optical system or optical head 11, which comprises a lens holder. The second end of reusable portion 101, which may be opposite the first end, may be closed and may comprise battery contacts, e.g., battery contacts 15a and 15b.

Reference is now made to FIG. 4, which illustrates a schematic back-side exploded perspective view of a reusable in-vivo device, in accordance with one embodiment of the present invention. In some embodiments, housing 10 of the reusable portion 101 of the reusable device may be inserted into a disposable portion 102. Disposable portion 102 may comprise at least a disposable shell 30 and a disposable optical dome 20. Housing 10 may be inserted into shell 30. Disposable optical dome 20 may then be attached to shell 30 in order to detachably seal the reusable portion 101 or housing 10 within disposable portion 102. For example, optical dome 20 may be screw threaded onto shell 30, or it may be snap fitted or friction fitted over a portion of shell 30, in order to later enable disassembly of optical dome 20 from shell 30. A combination of screw threading means and friction fitting means may be used.

An O-ring may also be attached to either shell 30 or optical dome 20. Thus, when screw threading or snap fitting optical dome 20 onto shell 30, the O-ring may provide good sealing of the device, so as to prevent entry of in-vivo fluids or other content into the device while the device passes through the lumen. The O-ring may be made of a variety of flexible materials, e.g., rubber or silicon. The O-ring may absorb the pressure created when optical dome 20 is pressed against shell 30 and may provide good sealing between the assembled optical dome 20 and shell 30. Other means of detachably sealing reusable portion 101 within disposable portion 102 may be used.

In some embodiments, optical dome 20 may be attached to shell 30 over optical system 11 of housing 10, in order to provide a transparent window through which light may pass from illumination source(s) 12 onto the tissue, and from the tissue through optical system 11 and onto imaging device 13. Optical dome 20 typically provides a space between optical system 11 and the lumen wall so that, if and when the tissue collapses over the optical dome, an image of the surrounding tissue may still be acquired.

In some embodiments, the reusable device may not comprise rechargeable batteries, thus housing 10 may not comprise rechargeable batteries. In such embodiments, disposable batteries may be inserted into disposable shell 30 before inserting housing 10 into shell 30. After inserting the disposable batteries into shell 30, housing 10 is inserted over it, and its electrical contacts 15a and 15b may be in contact with the disposable batteries. Disposable optical dome 20 may then be attached to shell 30 in order to detachably seal reusable portion 101 within disposable portion 102. In other embodiments, the disposable batteries may be an inseparable part of disposable portion 102, e.g., the disposable batteries may be an inseparable part of shell 30. This may ease the assembly process, since there is no need for an additional step of inserting the disposable batteries, but rather the disposable batteries may be inserted into reusable device 100 during the step of attaching optical dome 20 to shell 30.

Housing 10 may comprise the majority of relatively expensive components, e.g., optical system 11, illumination sources 12, imaging device 13, switch 17, PCB 16 that the electrical components are mounted on, and, in some embodiments, rechargeable batteries 18 and magnetic element 19. Disposable shell 30 may be made of plastic, e.g., polycarbonate, which is not considered an expensive material. Disposable optical dome 20 may also be made of plastic, e.g., polycarbonate, though optical dome 20 should be transparent, while shell 30 need not be transparent but may rather have an opaque color. Since housing 10, which comprises most of the relatively expensive components, is reusable, the reusable device 100 (such as shown in FIG. 5) that may be assembled from housing 10, optical dome 20 and shell 30 may be of a relatively low cost to the end-user. The costs of device 100 may be low, since housing 10 may be reused, while the cheap optical dome 20 and shell 30 are the only parts that need to be replaced between one use of device 100 and another use of device 100.

According to some embodiments, a controller (not shown) within device 100 may be used to predefine the number of uses of reusable device 100, after which device 100 will no longer work. In some embodiments, device 100 may be reused for a limited number of uses that may be restricted either by reduced functionality of the rechargeable batteries over time or by reduced functionality of other electrical components as a function of time. For example, the illumination sources 12 may have less intensity as time passes by, and, therefore, in order to acquire images of a decent quality, the number of reuses of device 100 should be limited. In some embodiments, every usage of the device may be counted by a counter, which may be part of the controller. During initiation process of device 100, a message indicating to the operator the remaining usages of device 100 may appear on a display unit (not shown), which may be connected to device 100. After the final reuse of device 100, the controller may shut down device 100 in order to prevent further usage that exceeds the predetermined number of reuses.

Reference is now made to FIG. 5, which illustrates a schematic back-side perspective view of a detachably assembled reusable in-vivo device, in accordance with one embodiment of the present invention. According to some embodiments, once housing 10 is inserted into the disposable portion 102 comprising shell 30 and optical dome 20, device 100 may be ready for use. Device 100 may be a reusable device, as disposable optical dome 20 is detachably attached to disposable shell 30. Therefore, after device 100 is placed within a lumen of patient and after it passes along the lumen, it may be collected or retrieved following its exit from the patient's body. Once the device 100 is collected by a physician or a nurse, it may be disassembled. Optical dome 20 may be detached from shell 30, housing 10 may be removed from within shell 30, and both optical dome 20 and shell 30 may be discarded. Housing 10 may then be inserted into a new disposable portion, i.e., housing 10 may be placed into at least a new disposable shell 30, which may have attached a new disposable optical dome 20 to detachably seal the reusable housing 10.

Device 100 may have a shape of an ellipsoid, a sphere, a capsule (e.g., cylinder capped with hemispheres) or any other shape that may be easily inserted into a patient's lumen, and which may easily and smoothly pass along the lumen.

In some embodiments, reusable device 100 may comprise rechargeable batteries, which may typically be encapsulated within housing 10. Therefore, prior to inserting the housing 10 into a new disposable portion, the rechargeable batteries may be recharged. In some embodiments, device 100 may comprise disposable batteries instead of reusable batteries. Thus, when disassembling device 100, the used disposable batteries may be discarded, and new disposable batteries may be inserted into the new disposable shell 30 prior to inserting the reusable housing 10. Once the disposable batteries are inserted into the new disposable shell 30, housing 10 may be inserted and a new disposable optical dome 20 may be attached to shell 30 in order to detachably seal the reusable housing 10. In some embodiments, the disposable shell 30 may comprise the disposable batteries (e.g., the disposable batteries may be an inseparable part of shell 30), and there is thus no need for a separate step of inserting the disposable batteries into the disposable shell 30 prior to insertion of reusable portion 101 into shell 30.

Device 100 may be retrieved by an operator (e.g., physician or nurse) after device 100 exits from the patient's body. A few methods may be used in order to retrieve or collect the device 100 at the end of the procedure. For example, if device 100 comprises maneuvering capabilities (e.g., using magnetic element 19) and if, for example, device 100 is passed along the GI tract, the operator may easily maneuver the device 100 through the rectum towards the anus and may collect device 100 as it leaves the patient's body. In some embodiments, the operator may collect device 100 with his hands, which may preferably be covered with gloves. In other embodiments, the operator may attach some sort of net member over the patient's anus, which may be used to collect the device 100 after it is maneuvered by the operator to exit the anus.

In yet other embodiments, device 100 may be collected by using “recovery equipment” for collecting capsule type endoscopes, such as disclosed in FIG. 14 of U.S. Pat. No. 7,856,676 to Toshimasa, et al., the entire contents of which are incorporated herein by reference. As can be seen therein, the capsule collector (61) may be mounted to the toilet stool (7) and sandwiched between the toilet stool (7) and the toilet seat (71) in order to fix the capsule collector (61). In such a manner, the collector (61) is prepared for discharging operation of the capsule (3) of the subject into the collector (61). When such a collector is used, device 100 may either be collected by the patient or by the operator of device 100.

Reference is now made to FIGS. 6A-6B, which illustrate a schematic view of a recharging unit before and during recharging of a reusable portion of an in-vivo device, respectively, in accordance with one embodiment of the present invention. When device 100 comprises rechargeable batteries, which may typically be encapsulated within housing 10, those batteries should be recharged after housing 10 is extracted from a used disposable portion 102 and before housing 10 is placed into a new disposable portion 102. An example of a recharging unit 200 for recharging rechargeable batteries (e.g., batteries 18, shown in FIG. 3C) is shown in FIGS. 6A-6B. According to some embodiments, recharging unit 200 may comprise a void 210, at an appropriate size for housing 10 to be inserted into. Typically, recharging unit 200 comprises a plurality of voids 210 in order to save time and simultaneously recharge a plurality of rechargeable batteries that are encapsulated in a plurality of separate reusable housings, which may be similar to housing 10.

In some embodiments, recharging unit 200 may comprise protrusion 240. Protrusion 240 may be complementary to groove 40, such that protrusion 240 may fit substantially precisely into groove 40. Groove 40 and its complementary protrusion 240 may provide specific orientation of reusable portion 1010 when inserted into recharging unit 200. A specific orientation of reusable portion 101 is important so that each of battery contacts 15a and 15b is positioned on top of its corresponding contact, which is placed within each of voids 210 of recharging unit 200. For example, battery contact 15a, which is the anode (the positive terminal), should be positioned on top of the positive terminal within void 210 of recharging unit 200, whereas battery contact 15b, which is the cathode (the negative terminal), should be positioned on top of the negative terminal within void 210 of recharging unit 200.

In other embodiments, instead of reusable portion 101 having two battery contacts, each located at a different location on the second end of reusable portion 101 (e.g., contacts 15a and 15b), reusable portion 101 may comprise two ring shaped battery contacts. The two rings may be concentric, such that their mutual center may be located in the middle of the second end of reusable portion 101 (opposite the end comprising the optical head 11), though other locations may be used. One of the ring shaped battery contacts may be the anode while the other one is the cathode. In this embodiment, recharging unit 200 may comprise two corresponding ring shaped contacts, such that, once reusable portion 101 is inserted into any of voids 210, the battery contacts of reusable portion 101 connect to their corresponding contacts within recharging unit 200. This embodiment makes groove 40 and complementary protrusion 240 redundant, and, therefore, there is no need for these two in such embodiments.

In some embodiments, recharging unit 200 may comprise an indicator to indicate that the rechargeable batteries are in the process of recharging, and in due time to further indicate when the recharging process is complete. In one embodiment, the indicator may comprise two illumination sources; illumination source 230 for indicating that the batteries (e.g., batteries 18) are in the process of recharging, and illumination source 220 for indicating that the recharging process is complete, i.e., that the batteries are fully recharged and may be reused following insertion into a new disposable portion 102. When illumination source 230 is lit, the operator may determine that the batteries are undergoing recharging process. Whereas, when illumination source 220 is lit, the operator may determine that the recharging process is complete, and the batteries or housing 10 may be removed from recharging unit 200, since the batteries are ready for reuse. In other embodiments, the indicator may comprise one illumination source that may illuminate at different colors; each color may indicate a different stage in the process of recharging the rechargeable batteries. For example, the same illumination source may illuminate in red color when the recharging process is ongoing, and may illuminate in green color when the recharging process is complete. In yet other embodiments, the indicator may be a sound indicator. For example, the recharging unit 200 may produce a sound, e.g., a beep sound, to indicate to the operator that the recharging process is complete. In other embodiments, other types of indicators may be used.

FIG. 6B illustrates housings 10 as having been placed within voids 210, such that the rechargeable batteries that are encapsulated within housing 10 may be recharged for re-use. Illumination sources 220 and 230 may be standard light emitting diodes (LEDs) or any other illumination sources. Typically, illumination sources 220 and 230 have a different color in order for the operator to easily notice the stage of recharging which the batteries are at.

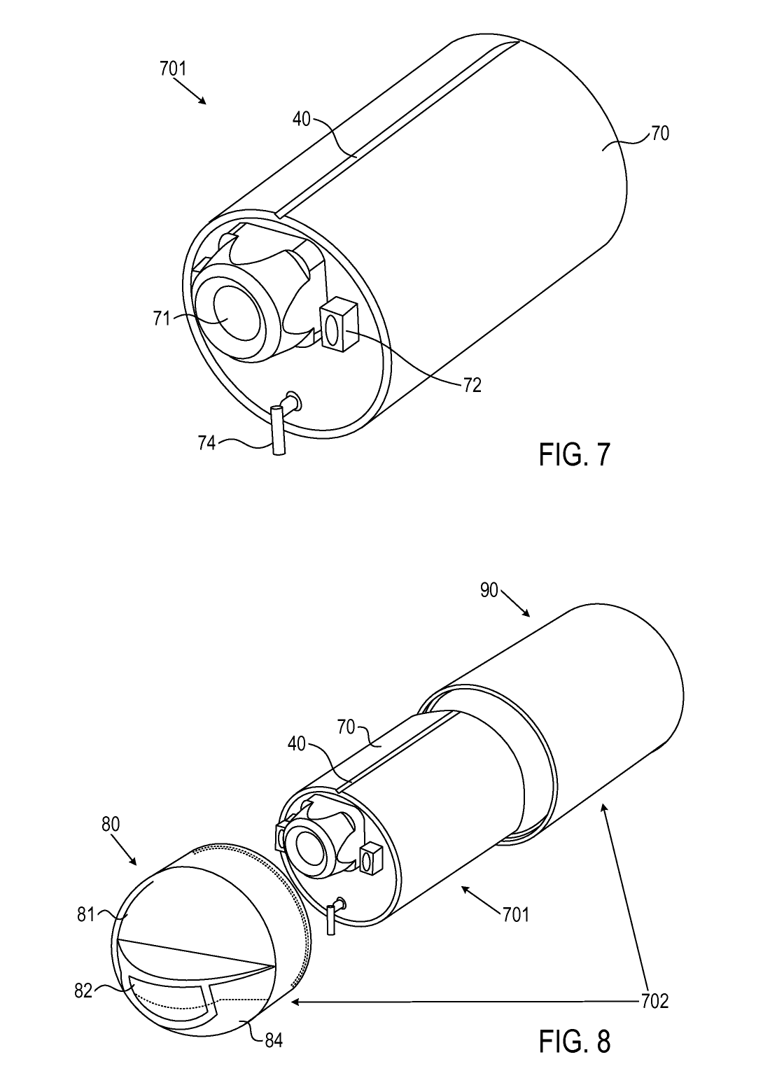

Reference is now made to FIG. 7, which illustrates a schematic front-side perspective view of a reusable portion of an in-vivo device, in accordance with another embodiment of the present invention. FIG. 7 illustrates an example of a reusable portion 701 of a reusable device that may perform biopsy while in-vivo. Housing 70 encapsulates similar components as those encapsulated in housing 10, though their arrangement may be somewhat different. Housing 70 may comprise an imaging device (not shown), an optical system 71, which may be placed over the imaging device, and at least one illumination source 72. All of these components may be mounted onto a PCB (not shown). In some embodiments, housing 70 may encapsulate rechargeable batteries (e.g., batteries 18), while in other embodiments, housing 70 need not contain rechargeable batteries but rather the electrical components encapsulated in housing 70 may be powered by disposable batteries.

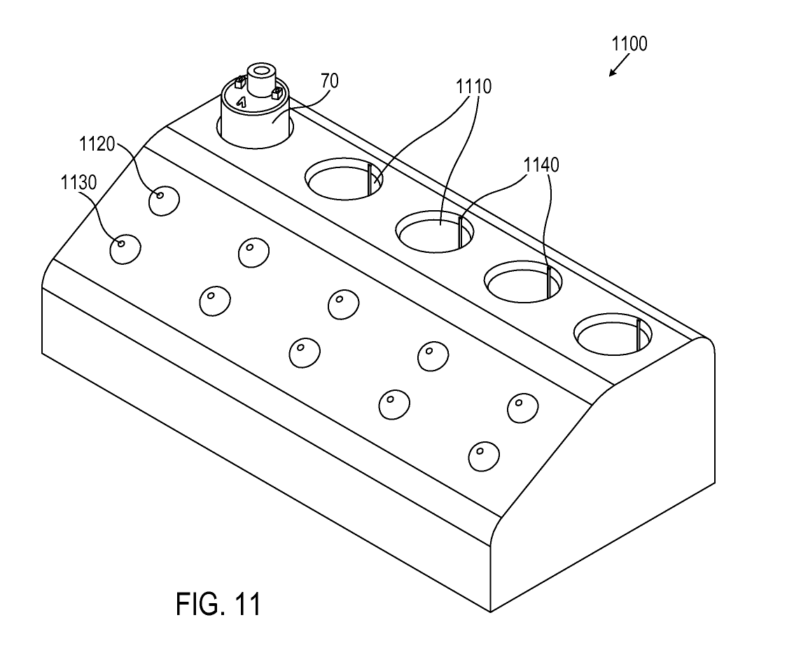

In some embodiments, housing 70 may comprise a groove 40 formed into its outer surface. Groove 40 may extend along substantially the entire longitudinal axis of housing 70, as shown in FIG. 7. In other embodiments, groove 40 may only partially extend along the longitudinal axis of housing 70. In yet other embodiments, groove 40 may extend along other positions of housing 70, e.g., along part of the transverse axis of housing 70. As will be described later with regards to FIG. 11, groove 40 may be used to place housing 70 into recharging unit 1100 at a specific orientation.

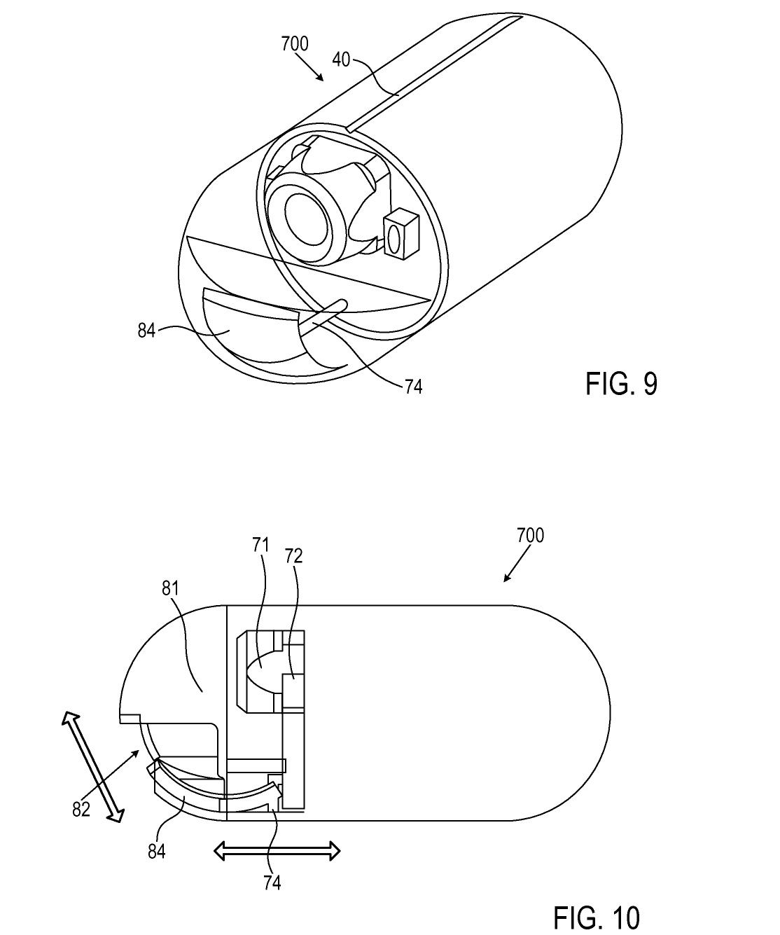

According to some embodiments, housing 70 may further comprise a driving arm 74, which may be pushed (e.g., by a motor) towards a biopsy knife 84 (FIGS. 8, 9) so that it pushes the knife 84 to cut off a tissue sample. Driving arm 74 may also be retracted inwards towards housing 70, after one biopsy sample cutting process is done, and in some embodiments, before another biopsy cutting process is initiated. Other tools may be incorporated within housing 70 or may be part of the disposable portion into which housing 70 may be inserted prior to administering the reusable device to a patient.

Reference is now made to FIG. 8, which illustrates a schematic front-side exploded perspective view of assembling a reusable in-vivo device, in accordance with an embodiment of the present invention. According to some embodiments, housing 70 may be inserted into a disposable portion 702 before being inserted into a lumen of a patient. The disposable portion 702 may comprise at least a disposable shell 90 and a disposable optical dome 80. Housing 70 may be inserted into shell 90, and optical dome 80 may be attached to shell 90 so as to detachably seal housing 70 within the disposable portion. For example, optical dome 80 may be screw threaded onto shell 90 or may be snap fitted over a portion of shell 90, in order to later enable disassembly of optical dome 80 from shell 90. The shape of the closed disposable portion 702, i.e., after shell 90 is attached to optical dome 80, may be the shape of an ellipsoid, a cylinder, a sphere, a capsule, or any other shape that may enable ease of insertion into the patient's body and ease of passage of the device through the body lumen.

According to some embodiments, optical dome 80 may comprise an area through which an image may be acquired, e.g., area 81. In some embodiments, optical dome 80 may comprise an additional chamber or area 82 through which a biopsy sample may be acquired. In some embodiments, optical dome 80 may further comprise a biopsy knife 84, which may be disposable as optical dome 80 is. According to some embodiments, a tissue sample or biopsy, may be acquired by the reusable device once the driving arm 74 pushes the biopsy knife 84 so as to close the opening in area 82, thereby cutting a tissue sample, which may then be kept within chamber or area 82. If the reusable device has maneuvering capabilities, the operator may press the opening in area 82 against a lumen wall so that some of the tissue is pushed into the opening in chamber or area 82. The operator may then trigger operation of the driving arm 74 to be pushed outwards from housing 70. Driving arm 74 may then push biopsy knife 84 to close in on the tissue that is located within the opening in chamber or area 82, thus biopsy knife 84 may cut the tissue, such that a sample of the tissue is now contained within chamber or area 82. After the reusable device is collected by the operator, the device may be disassembled, and the tissue sample contained within chamber or area 82 may be taken for examination in order to determine whether it contains any pathologies.

In some embodiments, area 82 may be separated from area 81 so that the tissue sample is kept apart from the imaging area 81, thereby not disturbing image acquisition.

In some embodiments, the reusable device may acquire more than one tissue sample either from the same area of interest or from a plurality of different areas of interest.

Housing 70 may contain the majority of relatively expensive components, e.g., optical system 71, illumination sources 72, imaging device (not shown), driving arm 74 and the motor (not shown) that pushes and/or retracts it, and PCB (not shown) onto which the electrical components may be mounted. In some embodiments, housing 70 may further comprise rechargeable batteries (not shown). Since housing 70 that contains the majority of relatively expensive components is intended to be reused, the device 700 (shown fully assembled in FIG. 9) may maintain a relatively low cost. Shell 90 and disposable optical dome 80 that comprises the biopsy knife 84 may be relatively low cost parts and may be discarded without significant loss. Shell 90 may be made of plastic, e.g., polycarbonate, which is not considered to be an expensive material. Optical dome 80 may be made of the same material as shell 90, though it should be transparent, while shell 90 may typically have an opaque color. The biopsy knife 84 may be made of plastic, metal or zirconia, or any other suitable material, as long as it is designed to have a sharp edge for cutting the tissue sample. Since biopsy knife 84 is of a small size, disposable optical dome 80 may still maintain a low cost, thus enabling the price of reusable device 700 to be reasonable to the end-user, e.g., the patient who is intended to use device 700.

Reference is now made to FIG. 9, which illustrates a schematic front-side perspective view of a detachably assembled reusable in-vivo device, in accordance with an embodiment of the present invention. FIG. 9 illustrates reusable device 700 after it has been assembled, e.g., after reusable housing 70 is placed within shell 90 and detachably sealed by optical dome 80. As seen from FIG. 9, the driving arm 74 pushes the biopsy knife 84 such that the opening in chamber or area 82 is sealed by the biopsy knife 84. Typically, driving arm 74 is activated by the operator after a tissue sample enters the opening in chamber or area 82. The sample may be kept within chamber or area 82 until the device 700 is collected by the operator. After device 700 exits the body lumen and after it is collected by the operator, it may be disassembled by the operator and the tissue sample contained within the chamber or area 82 may be sent to a pathology examination in order to determine the tissue sample's condition. Methods of collecting device 700 after it exits the patient's body may be similar to those described with regards to device 100 (FIG. 5).

Reference is now made to FIG. 10, which illustrates a schematic side view of a detachably assembled reusable in-vivo device, in accordance with an embodiment of the present invention. FIG. 10 illustrates reusable device 700 after it is assembled, such that housing 70 (not shown) is detachably sealed within shell 90 and optical dome 80. Optical dome 80 may comprise a first area 81 through which light from illumination source 72 may reach the lumen wall. Light may then be reflected from the tissue towards optical system 71 and onto an imaging device (not shown), which may be encapsulated within housing 70. In some embodiments, optical dome 80 may comprise a chamber or second area 82, through which a tissue sample may be acquired. The tissue sample may be kept within chamber or area 82 until device 700 exits the patient's body, which is when the biopsy sample may be extracted from chamber or area 82 and sent for examination.

Area 81 and chamber or area 82 may be separated from one another in order to prevent the acquired biopsy sample from blocking all or part of the field of view of the device 700, which may interfere with image acquisition. Furthermore, since area 81 contains electrical components, (e.g., illumination source 72), these components must be isolated from any in-vivo content (e.g., a tissue sample) that may cause short circuit.

Reference is now made to FIG. 11, which illustrates a schematic view of a recharging unit during recharging of a reusable portion of an in-vivo device, in accordance with an embodiment of the present invention. According to some embodiments, recharging unit 1100 may be similar to recharging unit 200 (shown in FIGS. 6A-6B). In some embodiments, recharging unit 1100 may comprise a void 1110 into which housing 70 may be inserted. Recharging unit 200 may typically comprise a plurality of voids 1110 in order to enable recharging process of a plurality of rechargeable batteries that may be contained within a plurality of housings 70, thus shortening recharging time.

In some embodiments, recharging unit 1100 may further comprise protrusions 1140, each of which may be located within each of voids 1110. Any protrusion 1140 may then fit into any of grooves 40 along housing 70 (as shown in FIG. 7) in order to ensure specific orientation of housing 70 when inserted into void 1110. The specific orientation of housing 70 within recharging unit 1100 may enable proper recharging of the reusable batteries encapsulated within housing 70, such that each of the battery contacts on housing 70 connects with its corresponding contact within void 1110 (similarly to the description of FIGS. 6A-6B above).

Recharging unit 1100 may further comprise an indicator to indicate that the rechargeable batteries are in the process of recharging, and in due time to further indicate when the recharging process is complete. In one embodiment, the indicator may comprise at least two separate illumination sources that, when lit, may indicate different stages of the recharging process. For example, when illumination source 1120 is lit, it may indicate that the process or recharging the rechargeable batteries has been completed, and that housing 70 may be removed from recharging unit 1100 and may be inserted into a new set of disposable optical dome 80 and disposable shell 90. When illumination source 1130 is lit, it may indicate that the recharging process is still ongoing, and that it is not yet the time to remove housing 70 from the recharging unit 1100. Illumination sources 1120 and 1130 may each have a different color, so as to ease distinction between the two, although in some embodiments, illumination sources 1120 and 1130 may have the same color. In other embodiments, the indicator may comprise one illumination source that may illuminate at a different color per each stage of recharging process of the rechargeable batteries. In yet other embodiments, the indicator may be a sound indicator, which may produce a sound indicating completion of recharging process. In other embodiments, other indicators may be used.

In other embodiments, housing 70 need not comprise rechargeable batteries, such that there is no need for recharging unit 1100. Instead, disposable batteries may be used and inserted into disposable shell 90 prior to the insertion of housing 70. Disposable optical dome 80 may then be attached onto shell 90, detachably sealing housing 70 and the disposable batteries within the disposable portion. The disposable batteries may be replaced between one usage of the device 700 to another, while replacing the shell 90 and optical dome 80 between uses.

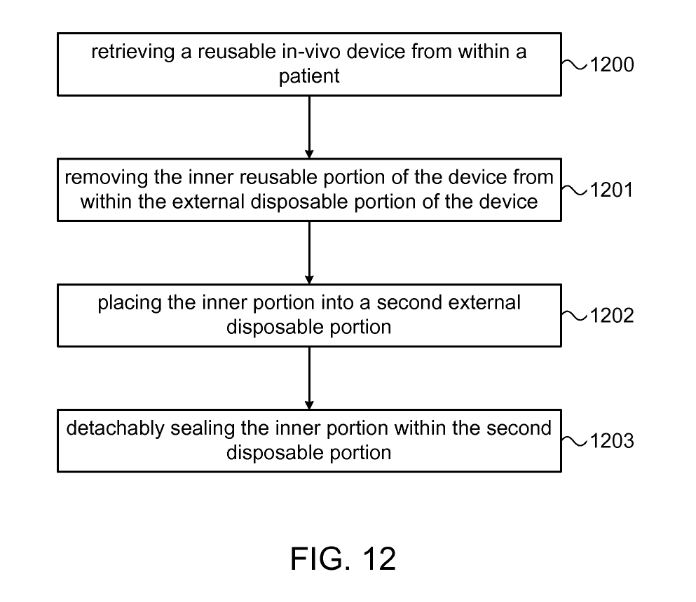

Reference is now made to FIG. 12, which illustrates a method of assembling a reusable in-vivo device, in accordance with an embodiment of the present invention. According to some embodiments, the method may comprise the step of retrieving a reusable in-vivo device from within a patient (1200). The reusable device may be similar to device 100 or 700 or may comprise other operational tools than the one used in device 700. The device may be inserted into a patient's lumen, e.g., the GI tract, and may either passively move along the lumen, or may be maneuvered along the lumen. The device may be inserted into the patient by swallowing or by using a delivery device that does not require the patient to swallow the device. In other embodiments, the device may be inserted into the patient's GI tract via the anus. For example, when the reusable device comprises maneuvering capabilities, another way of not requiring the patient to swallow the device may be by inserting it into the patient's body through the anus. The device may then be maneuvered from the anus to the colon, small intestine, stomach and so on, for examining and/or treating any of the various regions along the GI tract. The device may be inserted to other body lumens besides the GI tract, e.g., the reproductive tract, the urinary tract, or any other lumen that is easy for an in-vivo device to enter into and exit from.

Methods of retrieving the device may vary. If the device comprises maneuvering capabilities, e.g., the device comprises a magnetic element (e.g., magnetic element 19, FIG. 3B) and may be magnetically maneuvered by an operator, then the operator may maneuver the device towards the anus and either collect the capsule on his own, or may attach a net member over the anus to assist in retrieving the device. In some embodiments, the device may not comprise maneuvering capabilities, thus it may passively pass along the patient's lumen. In such embodiments, the patient may use a device collector such as the one disclosed in FIG. 14 of U.S. Pat. No. 7,856,676 to Toshimasa, et al., the entire contents of which are incorporated herein by reference.

Following retrieval of the device, the operator may execute the step of removing the reusable inner portion of the device from within the disposable outer portion of the device (1201). In order to reuse the reusable device, the operator may disassemble the device by removing the reusable inner portion, e.g., housing 10 or 70, from within the disposable outer portion, e.g., disposable shell 30 or 90 and disposable optical dome 20 or 80, respectively. After the reusable device is disassembled, the disposable portion may be discarded. In some embodiments, for example, when disposable optical dome (e.g., dome 80) comprises a biopsy sample, then the optical dome 80 that carries the sample may be sent to a pathologist's examination before it is discarded. In some embodiments, the method may comprise the step of detaching the shell of the device from the optical dome of the device, prior to removing the inner reusable portion from within the shell and dome.

Following disassembly of the device, the operator may perform the step of placing the inner portion into a second external disposable portion (1202). The operator may place the inner reusable portion (e.g., housing 10 or 70) into a new second set of disposable portion, e.g., into a new set of a disposable shell and a disposable optical dome. The method may further comprise the step of detachably sealing the inner portion within the second disposable portion (1203), such that the device may be ready for use in either a new patient or in the same patient but for a second time. According to some embodiments, detachably sealing the inner reusable portion within the external disposable portion may be done by screw threading the optical dome onto the shell, by snap fitting (with or without an O-ring) or by friction fitting the optical dome over the shell, thereby enabling disassembly of the reusable device after the reusable device is retrieved from within the patient. A combination of screwing means and snap fitting or friction fitting means may be used. Other means for detachably sealing the inner reusable portion within the external disposable portion may be used.

This method of retrieving the device, disassembling it, and reassembling it within a new second disposable portion may be performed many times, so as to reduce costs of an examination process by the reusable device. In some embodiments, the number of reuses of such a device may be limited to a predefined number of reuses, after which the device may be shut down by a controller. The number of reuses of the device may be restricted due to reduced functionality over time, e.g., reduced functionality of electrical components within the reusable inner portion as time passes by.

In some embodiments, the reusable device may comprise rechargeable batteries (e.g., rechargeable batteries 18). Therefore, the method may comprise a step of recharging at least one battery that is encapsulated within the inner reusable portion, (e.g., housing 10 or 70), prior to placing the inner portion into the second new disposable portion (e.g., disposable shell 30 or 90, and disposable optical dome 20 or 80, respectively). In other embodiments, the reusable device need not comprise rechargeable batteries. Therefore, the method of assembling a reusable in-vivo device may comprise the step of inserting at least one disposable battery into said second disposable portion (e.g., disposable shell 30 or 90, and disposable optical dome 20 or 80, respectively) prior to placing said inner reusable portion (e.g. housing 10 or 70) into the second disposable portion.

In some embodiments, the method may further comprise the step of inserting the detachably sealed reusable in-vivo device into a patient following the step of detachably sealing the inner reusable portion within the second disposable portion. The patient may either be a new patient who may undergo examination using the reusable device, or may be the same patient undergoing the same examination for a second time (or more). The device may acquire images of the in-vivo lumen, while passing through the lumen, either passively or by being maneuvered by an operator. In some embodiments, the device may perform various operations in addition to or without acquiring images of the lumen. For example, the device may be similar to device 700, which may comprise a biopsy knife 84 for acquiring a tissue sample from an area of interest. Other tools may be incorporated as part of the reusable device.

According to some embodiments, the method may comprise the step of maneuvering the reusable in-vivo device through a lumen of a patient, prior to retrieving the in-vivo device from within the patient. Maneuvering the reusable device may be done by, for example, magnetic maneuvering. According to some embodiments, the reusable device may encapsulate a magnetic element (e.g., magnetic element 19) and the operator may control an external magnetic field, e.g. change direction of the magnetic field, so as to control movement of the magnetic element within the reusable device and thus control movement and direction of movement of the device itself. In some embodiments, the reusable device may be maneuvered to an area of interest where in-vivo operations may be performed. Following retrieval of the device, disassembly of the reusable portion from the used disposable portion and reassembly in a second new disposable portion, the device may be inserted into a patient for an additional use. During the additional use, the device may be controlled again by the operator, such that it may be maneuvered along the in-vivo lumen, and may be held at a certain area for the purpose of performing some kind of examination and/or treatment while in-vivo.

While the present invention has been described with reference to one or more specific embodiments, the description is intended to be illustrative as a whole and is not to be construed as limiting the invention to the embodiments shown. It is appreciated that various modifications may occur to those skilled in the art that, while not specifically shown herein, are nevertheless within the scope of the invention.

1. An in-vivo device comprising:
a closed self-contained reusable inner portion comprising a housing, said housing encapsulating an imaging device, at least one illumination source, at least one rechargeable battery, and a controller, wherein a first end of said reusable inner portion is covered by an optical system, and wherein a second end of said reusable inner portion comprises battery contacts; and
a disposable outer portion comprising at least a shell and an optical dome,
wherein said housing of said reusable inner portion is inserted into said shell, and wherein said optical dome is attached to the shell so as to detachably seal the reusable inner portion of the in-vivo device within the disposable outer portion of the device.

2. The device according to claim 1, wherein said reusable inner portion further comprises operational tools for performing operations in-vivo.

3. The device according to claim 1, wherein the optical dome is detachably attached to the shell by means selected from a group consisting of: friction fit, screwing, or a combination thereof.

4. The device according to claim 3, wherein the means for detachably attaching the optical dome to the shell comprise an O-ring for sealing the optical dome to the shell.

5. The device according to claim 1, wherein said reusable inner portion further encapsulates a magnetic element for enabling maneuvering of the device using magnetic fields.

6. A system for assembling an in-vivo device, said system comprising:
an in-vivo device comprising:
a closed self-contained reusable inner portion comprising a housing, said housing encapsulating an imaging device, at least one illumination source, at least one rechargeable battery, and a controller, wherein a first end of said reusable inner portion is covered by an optical system, and wherein a second end of said reusable portion comprises battery contacts; and
a disposable outer portion comprising at least a shell and an optical dome,
wherein said housing of said reusable inner portion is inserted into said shell, and wherein said optical dome is attached to the shell so as to detachably seal the reusable inner portion of the in-vivo device within the disposable outer portion of the device; and
a recharging unit for recharging the rechargeable battery encapsulated within the reusable inner portion, thereby enabling reuse of the reusable inner portion.

7. A method of assembling an in-vivo device, said method comprising:
retrieving an in-vivo device from within a patient, said device comprising a closed self-contained reusable inner portion and a disposable outer portion, wherein said reusable inner portion encapsulates an imaging device, at least one illumination source, at least one rechargeable battery, and a controller, wherein a first end of said reusable inner portion is covered by an optical system, and wherein a second end of said reusable inner portion comprises battery contacts, and wherein said disposable outer portion comprises at least a shell and an optical dome;
removing the reusable inner portion of the device from within the shell and optical dome;
placing said reusable inner portion into a second disposable outer portion, wherein said second disposable outer portion comprises at least a second shell and a second optical dome; and
detachably sealing the reusable inner portion within the second disposable outer portion.

8. The method according to claim 7 comprising the step of detaching the shell of the device from the optical dome of the device, prior to removing the reusable inner portion from within the shell and dome.

9. The method according to claim 7 comprising the step of recharging the at least one rechargeable battery, prior to placing the reusable inner portion into the second disposable outer portion.

10. The method according to claim 7 comprising the step of inserting the detachably sealed in-vivo device into a patient following the step of detachably sealing the reusable inner portion within the second disposable outer portion.

11. The method according to claim 7 comprising the step of maneuvering the in-vivo device through a lumen of a patient, prior to retrieving the in-vivo device from within the patient.
